(19) United States
(12) Patent Application Publication
(10) Pub. No.: US 2006/0135869 A1
(43) Pub. Date: Jun. 22, 2006

(54) System and method for measuring arterial vessels using near infrared spectroscopy at simultaneous multiple wavelengths

(76) Inventor: James D. Farina, Howell, NJ (US)

Correspondence Address: Glen M. Diehl;Norton & Diehl LLC, Suite 110, 77 Brant Avenue, Clark, NJ, 07066, US

(21) Appl. No.: 11/293,938

(22) Filed: Dec. 5, 2005

Related U.S. Application Data
(60) Provisional application No. 60/681,484, filed on May 16, 2005.
(60) Provisional application No. 60/633,934, filed on Dec. 7, 2004.

(51) Int. Cl. A61B 5/05

(52) U.S. Cl. 600/421

(57) ABSTRACT

A system and method for improving the examination of vessel walls through fluid using near infrared (NIR) spectroscopy by employing a parallel measurement where all wavelengths are measured simultaneously. The system and method of the present invention obviates that need to attempt to overcome the motion of a catheter by complex filtering or averaging over time by performing the measurements for each wavelength under identical conditions.

### STATEMENT OF RELATED CASES

This application claims the benefit of U.S. Provisional Patent Application Ser. No. 60/633,934 filed Dec. 7, 2004 and also claims the benefit of U.S. Provisional Patent Application Ser. No. 60/681,484 filed May 16, 2005, both of which are hereby incorporated herein by reference in their entirety.

### BACKGROUND OF THE INVENTION

Arterial catheter-based systems are employed in the identification of atherosclerotic lesions or plaques that are characteristic of an arterial disorder involving the walls of medium- or large-sized arteries, including the aortic, carotid, coronary, and cerebral arteries.

For example, in one spectroscopic application, an optical source is used to access or scan a spectral band of interest between 750 nm and 2500 nm. This light is used to illuminate tissue in a target area in vivo using the catheter. Diffusely reflected light resulting from the illumination is then collected and transmitted to a detector system, where a spectral response is resolved. The response is used to assess the state of the tissue.

Difficulty arises because of the presence of intervening fluid, e.g. blood, and the relative motion of the arterial wall and the probe/catheter. Because the intervening fluid can pose its own spectral characteristics, due to absorption, scattering and fluorescence, the time varying distance between the probe and the vessel wall can result in significant masking of the reflected light from the vessel wall. Performing the measurement serially or one spectral region at a time will result in spectral measurements with potentially large enough variations so as to make accurate and reliable conclusions difficult or impossible.

Accordingly, new and improved system and methods to measure arterial vessels, and in particular, plaque in arterial vessels, are needed.

### SUMMARY OF THE INVENTION

The present invention concerns the improvement of the examination of vessel walls through fluid, such as blood. In the specific example, the invention is used for near infrared (NIR; 750-2500 nm) spectroscopy. NIR spectroscopy of the vessel walls by monitoring the spectral characteristic of the diffusely reflected light is complicated by the presence of blood between the probe and the surface itself. Blood can degrade the reflected signal by absorption and scattering, thus attenuating the reflected signal. Further complicating the spectral measurement is the fact that during the measurement, the distance between the vessel wall and thus the amount of blood to be traversed varies as a function of time. The distance variation is the result of the cardiac cycle, breathing or even the manipulation of the catheter in the vessel.

In accordance with one aspect of the present invention, the spectral measurements are performed simultaneously, thus synchronizing any signal impairments in the individual spectral regions being measured. One aspect of the present invention employs the encoding of each of the spectral portions of the illumination sources with unique tags or identifiers that allows for the separation of the spectral signals in the detection and analysis portions of the system.

Performing the spectral measurements in a parallel fashion, thus measuring all of the spectral regions of interest simultaneously, largely eliminates the existence of different variations in each of the spectral regions because they are all measured simultaneously and therefore suffer synchronized degradations during the measurement. This method eliminates the need for complex adaptive filtering and signal processing techniques to remove unsynchronized signal degradations. Additionally, this method allows for the measurement of spectral information over complete cardiac cycles or other time varying processes resulting in differing spacing between the probe and the vessel walls.

The present invention does not require that the probe periodically or even repeatedly approach the vessel wall. Nor does it require the knowledge of the position of the probe relative to the wall.

Thus, the present invention improves the examination of a vessel wall using a NIR spectroscopic method that allows for the parallel or simultaneous measurement of all spectral components. One such approach employs the encoding of each of the spectral portions of the illumination sources with unique tags or identifiers that allows for the separation of the spectral signals in the detection and analysis portions of the system.

Performing the spectral measurements in a parallel fashion, thus measuring all of the spectral regions of interest simultaneously, largely eliminates the existence of different variations in each of the spectral regions because they are all measured simultaneously and therefore suffer synchronized degradations during the measurement. This method eliminates the need for complex adaptive filtering and signal processing techniques to remove unsynchronized signal degradations. Additionally, this method allows for the measurement of spectral information over complete cardiac cycles or other time varying processes resulting in differing spacing between the probe and the vessel walls.

Not only does the present invention result in the automatic and natural synchronization of signal impairments in all spectral regions but also enables the measurement to be performed faster by a factor equal to the number of spectral regions of interest. For example, if there are 10 spectral regions of interest, a parallel or simultaneous measurement as described by this patent will require one tenth ( 1/10) of the time required by a conventional serial NIR measurement as is the case for tunable source spectroscopic techniques. The present invention does not require that the probe periodically or even repeatedly approach the vessel wall. Nor does it require the knowledge of the position of the probe relative to the wall.

In general, according to one aspect, a method is provided for optically examining blood vessel walls with a probe through intervening fluid, the method comprising:

illuminating the vessel walls with multiple spectral sources;

receiving optical signals from the vessel walls through the intervening fluid at the probe; and

analyzing the optical signals to determine the wavelength dependency of the reflectivity of the vessel wall.

In general, according to another aspect, a system is provided for optically examining blood vessel walls with a probe through intervening fluid, the system comprising:

a probe to illuminate the vessel walls with multiple spectral sources;

a detector to receive optical signals from the probe;

and a processor to analyze and measure the spectral information.

In the preferred embodiment, the reflected light signals are collected and transported back to a single detector for conversion to electrical signals and subsequent separation of the spectral regions.

In another embodiment of the present invention, the reflected light signals are converted to an electrical signal by a detector housed inside the probe/catheter and then transmitted back to the signal processing system where the separation of the spectral region information is performed.

The present invention allows for uniform and non-uniform signal impairments across the various spectral regions. Thus, if one spectral region is affected by the intervening fluid differently than another region for the same environmental conditions (e.g. probe to wall separation), the effect of the impairment can be removed by simple scaling without the need for any re-synchronization in time.

In still other embodiments, the methods and systems of the present invention are used for identifying vulnerable plaques in a subject and diagnosing subjects at risk for acute coronary events, such as unstable angina, myocardial infarction, and sudden cardiac death.

Other features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

FIG. 1 is an illustration of a preferred embodiment showing the amplitude modulation of the spectral sources, fiber launch and collection and processing of the reflected light signals.

FIG. 2a is an illustration and an example of the reflection spectrum for a substance of interest.

FIG. 2b is an illustration of the different source spectral regions chosen for the example of FIG. 2a.

FIG. 2c is an illustration of the overlap of the reflection spectrum and the source spectral regions from FIGS. 2a and 2b.

FIG. 3 is an example of uniform impairments on a reflected spectrum from a substance of interest under different separations of the probe and vessel wall in the presence of an intervening fluid.

FIG. 4a is an example of non-uniform impairments on a reflected spectrum from a substance of interest under different separations of the probe and vessel wall in the presence of an intervening fluid.

FIG. 4b is an illustration of the source spectral regions with the addition of a spectral region to monitor the strength of the non-uniform impairment of FIG. 4a.

FIG. 5 is an illustration showing the elimination of electrical filtering in a preferred embodiment.

FIG. 6 is an illustration showing the implementation of a preferred embodiment with the detector moved into the probe/catheter.

FIG. 7 is an illustration showing a technique for simultaneously measuring multiple samples or multiple areas of a single sample.

### DETAILED DESCRIPTION OF THE INVENTION

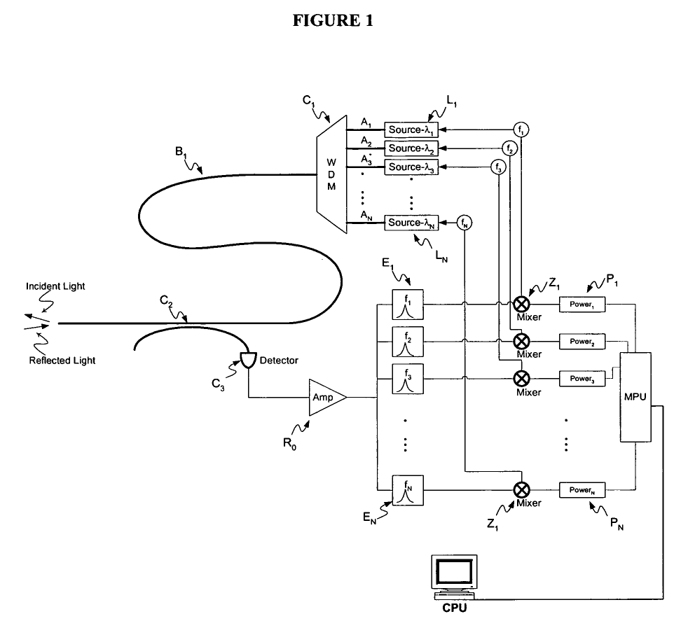

One implementation of the method and system of the present invention is shown in FIG. 1. In this arrangement, optical sources L₁ through L_{N} are used to supply the specific wavelengths that are to be observed. Each of these sources is amplitude modulated by varying the drive current or voltage at a unique frequency, F₁ through F_{N}. The frequencies are provided by a plurality of frequency sources f₁ through f_{N}.

The light output from these modulated sources is then launched onto fibers A₁ through A_{N}. The optical energy from these sources that is provided on fibers A₁ through A_{N} is then combined in a Wavelength Division Multiplexer C₁ to form a single optical signal comprised of all of the modulated light source outputs.

This composite optical signal is then carried in optical fiber, B1, through optical coupler C₂ to the measurement area. The composite optical signal is output from the fiber to the measurement area. In accordance with a preferred embodiment of the present invention, the measurement area is a blood vessel.

In the measurement area, the composite optical signal is incident on the sample under test, and where a reflected signal is generated. This sample may have a reflectivity that is different for each optical wavelength. Therefore, the amount of the incident optical signal that is reflected back into the measurement system can differ from wavelength to wavelength comprising the composite optical signal.

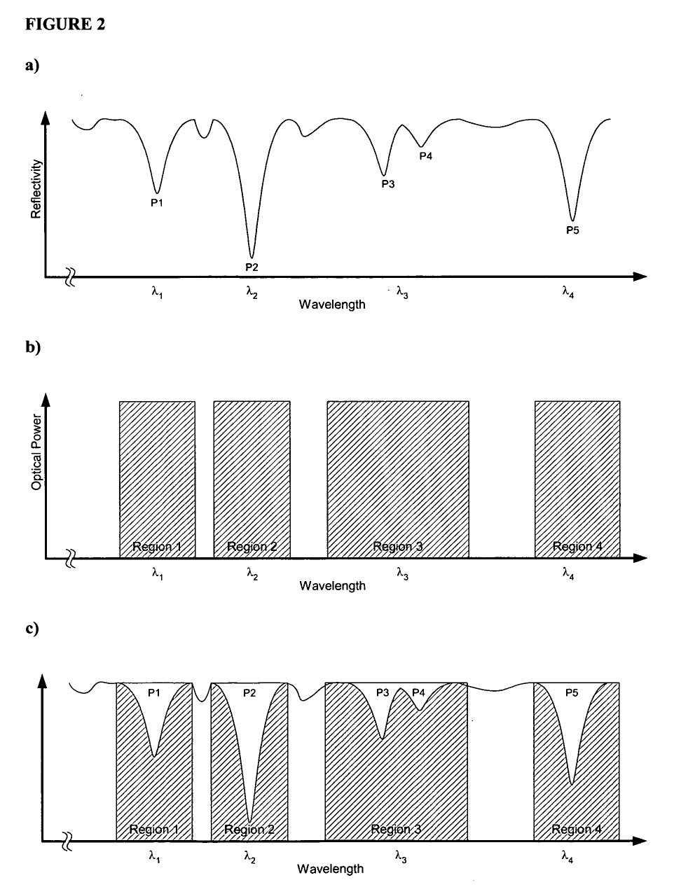

As an example of the choice of source regions, FIG. 2a shows an illustration of the spectral signature of the reflectance of a substance of interest. In this spectral signature, five reflection minima are identified as P1 through P5. FIG. 2b shows an illustration of the choice of spectral regions representing the spectrum of the sources. In this case, four (4) spectral regions of interest are chosen and FIG. 2c illustrates the overlap of the source spectral regions and the reflection minima. Thus, by measuring the reflectance in each of these regions and processing the resulting data by examining the ratios of the reflectance values of each region, the likelihood of the presence of the substance of interest on the vessel wall can be assessed. In accordance with a preferred embodiment of the present invention, the substance of interest is plaque.

At least some of the reflected optical signal is picked up by the fiber B1. Upon return to the measurement system, the composite reflected optical signal is then, as shown in FIG. 1, carried in the reverse direction in optical fiber, B₁, to an optical coupler C₂. A portion of the composite reflected signal is then coupled out of fiber B₁ and into an optical detector C₃ where it is converted into an electrical signal.

Because each of the optical sources are amplitude modulated at a unique frequency, the electrical spectrum emerging from the detector, C₃, will be made up of a collection of carriers at frequencies, f₁ through f_{N}. The amplitude of these carriers will be proportional to the reflectivity of the sample under test at the wavelength corresponding to the source modulated at the carrier frequency. In FIG. 1, the detector C₃ is located adjacent the transmitter and receives the reflected optical signal after it travels along the fiber B₁. The detector C₃ can also be located at the other end of the fiber B₁, where the reflected optical signal is picked up.

In this embodiment, the electrical signal from the detector C₃ is then amplified by amplifier R₀ and then passed to a series of electrical bandpass filters, E₁ through E_{N}, having center frequencies, f₁ through f_{N}, respectively. The outputs of the filters E₁ through E_{N} are fed into a coherent detector comprised by the mixers Z₁ through Z_{N}. In this embodiment, the mixers, Z₁ through Z_{N}, perform a multiplication of the electrical signal and the reference signal from the modulating oscillators of frequency f₁ through f_{N}. The output of the mixer is a DC level proportional to the amplitude of the electrical carrier at the frequency corresponding to the reference signal frequency. Thus, the DC level at this point is proportional to the reflected optical signal amplitude. Each channel of the detection system is a measurement of the reflectivity of the wavelength being modulated at the frequency corresponding to the particular channel's reference frequency.

After detection, scaling and filtering can be performed by the circuits P₁ through P_{N}, and then an electrical signal can be passed to a processor MPU where the normalization and other algorithms are performed to further process this spectral information.

Further processing and display are performed by a processor CPU connected via an interface provided to MPU.

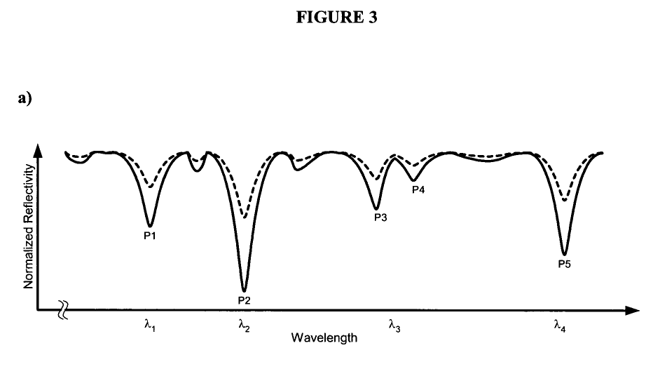

Simple ratiometric analysis of the spectral components could be used to eliminate the effect of the intervening fluid, blood, if the effect is uniform across the entire spectrum as illustrated in FIG. 3.

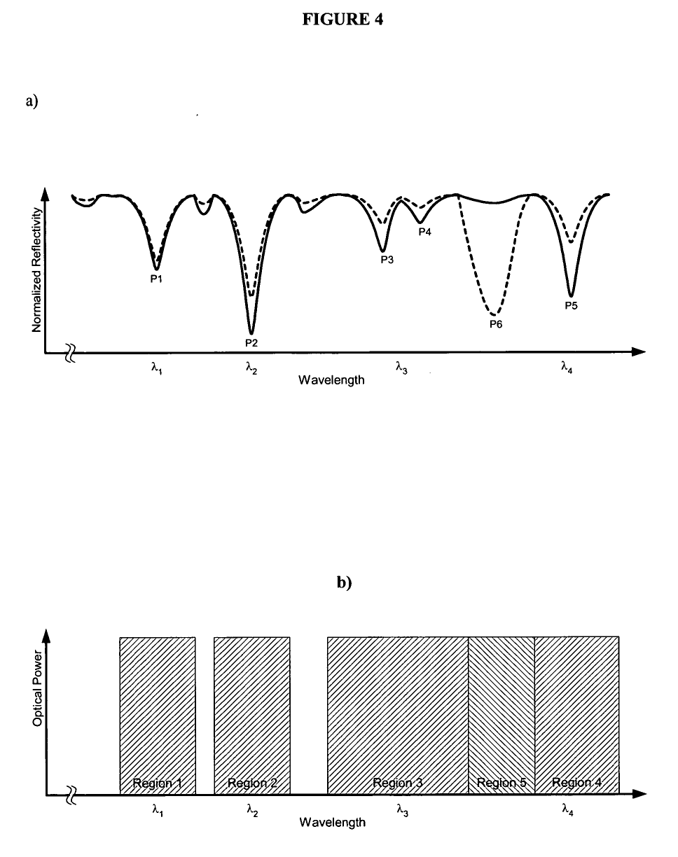

In the case where the effect of the intervening fluid, blood, is non-uniform as illustrated in FIG. 4a, another spectral region, Region 5 as shown in FIG. 4b, could be identified as an indication of the strength of the impairment imposed by the intervening fluid. Under these conditions, the reflectivity of light in Region 5 could be used as a scaling factor which is different for each spectral region. Thus, a correction for the impairment can be implemented before the ratiometric analysis is performed.

The processor MPU can be any type of processor, including, without limitation, a microprocessor circuit or a gate array circuit. The processor MPU is preferably connected to a display CPU. The CPU can be connected during use of the system of FIG. 1 on a patient or it can be connected at a later time for future analysis. The processor CPU also preferably includes a storage device, such as a hard drive or an optical disk drive to store data receive and/or analysis performed for future use.

The processing and analysis of the data can be performed entirely by the CPU connected to the detection subsystem via an interface provided by the processor MPU.

The processing of data when testing a patient for plaque in arteries is known. For example, processing sequentially received optical signals is known and those processing techniques can be applied to the present system and method. The processor can perform numerous types of processing, including analyzing a spectral response of the optical signals based on spectral features of the intervening fluid, analyzing the optical signals by performing an algebraic analysis of the spectral response, performing an algebraic analysis that includes a ratiometric comparison of the spectral response at multiple wavelengths, comparing the spectral response of the optical signals to known spectral features of blood, analyzing a difference in the spectral response at multiple wavelengths, comparing the spectrum of the optical signals to the spectral response of the intervening fluid.

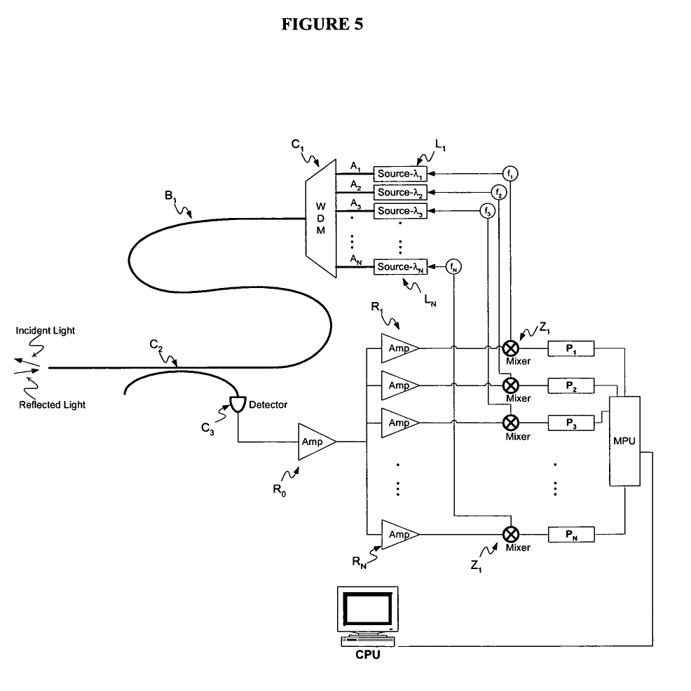

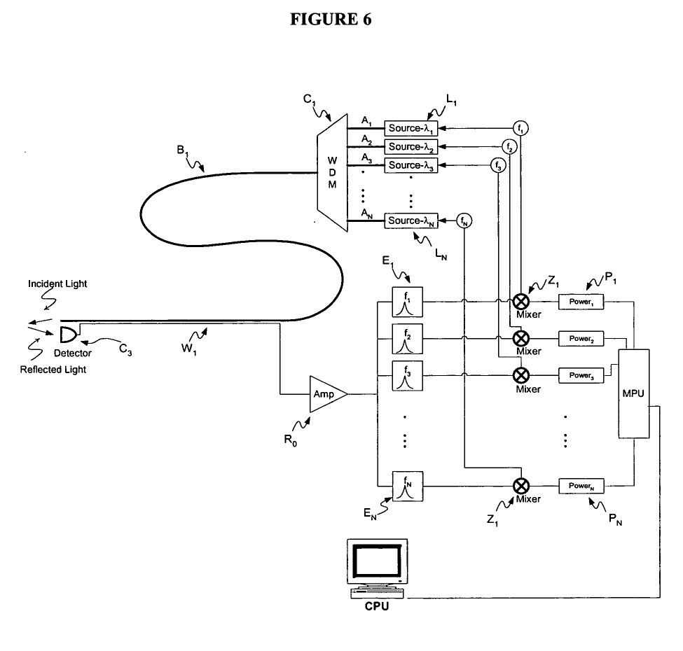

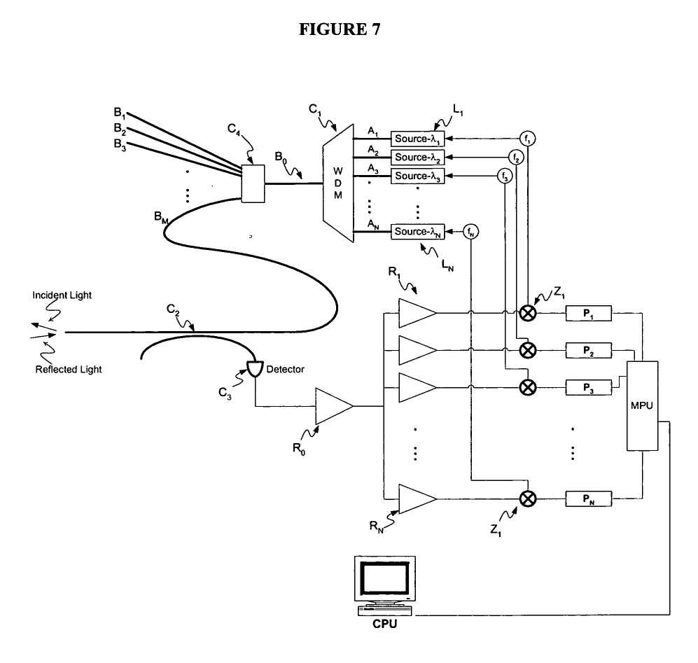

Variations of the embodiment of FIG. 1 include the arrangements shown in FIG. 5, FIG. 6 and FIG. 7. In FIG. 5, the transmission system includes a plurality of optical sources L₁ through L_{N}, a plurality of frequency sources f₁ through f_{N}, optical fibers A₁ through A_{N}, a Wavelength Division Multiplexer C₁ and an optical fiber, B1. These components function the same way as previously described with respect to the system of FIG. 1.

In the receiving section of the system of FIG. 5, a pick-up device C₂, a detector C₃ and an amplifier R₀ are provided. These components operated in a manner similar to the corresponding components of FIG. 1. The amplifier R₀ provides the signals to a parallel set of amplifiers R₁ to R_{N}. The signals are then fed into a coherent detector comprised by the mixers Z₁ through Z_{N} that perform a multiplication of the electrical signal and the reference signal from the modulating oscillators of frequency f₁ through f_{N}. As was the case with the circuit of FIG. 1, the output of the mixer is a DC level proportional to the amplitude of the electrical carrier at the frequency corresponding to the reference signal frequency. The signals from the coherent detector are fed to post-filtering circuits P₁ through P_{N} and then to a processor circuit MPU. The processor circuit MPU can be connected to processor CPU containing a display and storage device, as before.

In the circuit of FIG. 5, the bandpass filters are replaced with amplifiers R₁ through R_{N}. Thus, the burden of filtering is placed on the post filtering stage P₁ through P_{N}.

FIG. 6 illustrates another variation in accordance with another aspect of the present invention in which an optical detector is placed inside the probe. Thus, the optical detector is located inside a patient's blood vessel when the system is in use. In this case wires, W₁, transport the electrical signals from the optical detector, C₃, back to the signal processing system. The signal processing system can be identical to the other embodiments disclosed herein employing a fiber to transport the reflected optical signal.

FIG. 7 is an arrangement that could be used to simultaneously measure multiple samples or multiple areas of a single sample. This arrangement utilizes an optical splitter C₄ to split the composite optical source signal to feed the measurement and detection systems.

Variations of the embodiment include the use of alternative modulation techniques other than amplitude modulation at discrete frequencies. This includes the use of complex rf spectral signatures comprised of unique stationary or non-stationary combinations of frequencies impressed upon the optical sources so as to allow for the identification of the reflected optical source signals.

Further variations in the make up of the modulation format could include the use of a unique orthogonal digital code impressed on each optical source so the through the use of the appropriate decoder the reflected optical source signals may be identified and separated.

The methods and systems of the present invention find utility in the identification of atherosclerotic lesions or plaques that are characteristic of various arterial disorders. In particular, the method and systems of the present invention are particularly suited to the identification of so-called “vulnerable” plaques, as well as the diagnosis of subjects at risk for acute cardiac events. The concept of vulnerable plaque represents a significant departure from the conventional wisdom that assumed the most severely stenosed areas to be the most dangerous areas in an artery.

Vulnerable plaque, also referred to as “dangerous”, “unstable” or “at-risk” plaque, is commonly defined as plaque having a lipid pool with a thin fibrous cap, which is often infiltrated by macrophages. Vulnerable plaque lesions generally manifest only mild to moderate stenoses, as compared to the large stenoses associated with fibrous and calcified lesions. While the more severe stenoses of fibrous and calcified lesions may limit flow and result in ischemia, these larger plaques often remain stable for extended periods of time. In fact, rupture of vulnerable plaque is believed to be responsible for a majority of acute ischemic and occlusive events, including unstable angina, myocardial infarction, and sudden cardiac death.

The mechanism behind such events is believed to be thrombus formation upon rupture and release of the lipid pool contained within vulnerable plaque. Thrombus formation leads to plaque growth and triggers acute events. Plaque rupture may be the result of inflammation, or of lipid accumulation that increases fibrous cap stress. Clearly, prospective identification and stabilization of vulnerable plaque is key to effectively controlling and reducing acute ischemic and occlusive events.

A significant difficulty encountered while attempting to identify and stabilize vulnerable plaque is that standard angiography provides no indication of whether or not a given plaque is susceptible to rupture. Furthermore, since the degree of stenosis associated with vulnerable plaque is often low, in many cases vulnerable plague may not even be visible using angiography. Thus, techniques are needed which are able to detect the unstable atherosclerotic plaque, independent of the degree of luminal diameter narrowing, and treat it before unstable angina and/or acute myocardial infarction and its consequences occur.

In this respect, NIR spectroscopy has recently been used to detect lipid pool, thin fibrous cap, and inflammatory vulnerable plaques in nonstenotic vulnerable plaques, both in vitro (Moreno et al., Circulation 105:923 (2002); Wang et al., J. Am. Coll. Cardiol. 39:1305 (2002)) and in vivo (Moreno and Muller, J. Interv. Cardiol. 16:243 (2003)).

Because the methods and systems of the present invention measure all of the spectral regions of interest simultaneously, they are better suited to identifying vulnerable plaque through intervening fluid, e.g. blood., than prior NIR methods. Thus, another object of the present invention is to provide a method of identifying vulnerable plaque in a subject, such as a human or animal, by optically examining a vessel or body cavity wall as described above. The processor is programmed with algorithms and calibration data readily available to those skilled in the art allowing it to analyze the reflected spectral signals, wherein the output categorizes the scanned vessel tissue as either healthy or a vulnerable plaque.

The digital data can be processed using any or a variety of discrimination algorithms (qualitative analysis) to determine the nature of the correlation between the constituents within the blood vessel walls (as determined by an external means such as morphometry measurements or chemical analysis) and the spectral features obtained in the NIR spectrum (the digital data).

In some embodiments, once a metric has been chosen, a threshold is applied to determine the likelihood of whether the unknown tissue spectrum can be classified as a diseased tissue type or not. Many methods can be used for this determination such as a simple wavelength comparison technique using linear regression lines, or more complex geometries such as Euclidean or Mahalanobis distances as thresholds for more complicated separations.

In other embodiments, the original processed data (in the form of a set of numbers, with one number for each point or location within a scanned tissue sample) is continuously graded using standard techniques to provide a scale or value for each point without the use of a threshold. Thus, these methods utilize the raw scores, or the so-called “discriminant” based on the detected radiation, directly to provide a continuous scale, rather than comparing the discriminant to a threshold and providing a “yes/no” or other similar answer based on specific categories.

By using a continuous scale to represent the set of numerical data representing the scanned locations within a tissue, e.g., in an artery, the new methods and systems can provide the user, e.g., a physician, nurse, or technician with the opportunity to diagnose the vulnerability of a particular lesion without a threshold, and thus without the risk of an improperly set threshold, which could cause an incorrect diagnosis.

Another advantage of the thresholdless display is that the operator can make his or her own decisions as to the trade-off between sensitivity and specificity, by applying his or her own categories, criteria, or thresholds (which would otherwise be dictated by the system). The thresholdless display enables the operator to review a variety of discriminant values from multiple locations within a given patient, and compare those values to each other to make a diagnosis.

In some embodiments, the threshold and continuous grading techniques can be used together to provide a double-checking system.

Not only do the methods described herein enable collection of data relevant to detecting vascular lesions, such as vulnerable plaques, through blood within a living subject, it permits the operator to characterize the lesion, i.e., to determine whether a detected plaque is “vulnerable,” i.e., likely to rupture, or “safe,” i.e., unlikely to rupture. More specific characterization is also possible. The spectra received from the blood vessel walls can be analyzed by taking point readings and determining whether the location of the vessel wall corresponding to that point reading is predominantly lipid with a thin cap (vulnerable or “life-threatening”), lipid with a thick fibrous cap (potentially vulnerable), or predominantly non-lipid, normal, fibrotic, or calcific (safe or “non-life threatening”). Thus, the operator can create two (vulnerable/diseased or safe/healthy), three (vulnerable (diseased), potentially vulnerable (diseased), or safe (healthy)), or more different categories for lesion types. Alternatively, the system can provide a continuously graded output for the operator to decide whether a particular tissue is normal or has a lesion that is vulnerable or safe, without the use of a threshold.

In addition to the largely qualitative analysis discussed above, quantitative analysis can be used to determine the actual concentration of specified chemical constituents retained within a given location of tissue or lesion. For example, spectral information can be directly linked to the actual chemical constituent using a variety of different types of quantitative analysis based upon both univariate and multivariate analysis techniques. In this way, the methods of the present invention can be used to identify the chemical content of the lesion directly or, for example, in the form of a percentage of lipid, fibrotic, calcific, cholesterol, macrophage, or water content within the illuminated area. The methods can also be used to determine the pH or temperature of the diseased tissue or blood.

Although the methods and systems provided herein may be used to detect vulnerable plaques in subjects prior to their first acute cardiac event, most individuals actually experience their first event prior to their first catheterization. Accordingly, the methods described herein may be performed on patients undergoing percutaneous transluminal coronary angioplasty (PTCA)/stenting who will already have a wire inserted in a culprit artery for clinical reasons. A rationale for NIR imaging in these patients is that during the year following PTCA/stenting, approximately 10% will experience death, myocardial infarction, or require repeat revascularization because of rapid progression of plaque other than the one originally treated. It is the progression of such plaques that has substantially led to the inability of PTCA/stenting and coronary artery bypass grafting (CABG) to prevent subsequent MI in randomized studies.

Once a lesion or plaque is detected and determined to be vulnerable (or diseased), various technologies can be used for removing or stabilizing the plaque before it ruptures. For example, lasers can be used to ablate the plaque. Alternatively, one can use brachytherapy, angioplasty, stenting (coated or not), and photodynamic therapy. In addition, different therapies have been developed thus far for stabilizing vulnerable plaques. These include lipid lowering drugs (e.g., statins), matrix metalloproteinase (MMP) inhibitors, and sPLA2 inhibitors. These and future treatments may be carried out in light of the benefits conferred by the invention as described herein.

Thus, in accordance with one aspect of the present invention, a method for optically examining blood vessel walls with a probe through intervening fluid is provided. The method includes simultaneously illuminating the vessel walls with multiple spectral sources, receiving optical signals from the vessel walls through the intervening fluid at the probe, and analyzing the optical signals to determine the wavelength dependency of the reflectivity of the vessel wall.

The optical illumination from the probe can be comprised of multiple spectral sources each with a unique amplitude modulation allowing for the separation of spectral information subsequent to reflection from the vessel walls.

The step of analyzing the optical signals can include the separation of the different spectral signals and determining their respective amplitudes and can also include analyzing the optical signals comprises analyzing a spectral response of the optical signals based on spectral features of the intervening fluid. The step of analyzing the optical signals can further include performing an algebraic analysis of the spectral response. The algebraic analysis can include a ratiometric comparison of the spectral response at multiple wavelengths.

The intervening fluid between the wall and the instrument is generally blood and the method, in accordance with one aspect of the present invention, further comprises comparing the spectral response of the optical signals to known spectral features of blood. All of the analysis steps previously mentioned can be used for this spectral analysis as well.

The algebraic analysis can include analyzing a difference in the spectral response at multiple wavelengths. It can also include comparing the spectrum of the optical signals to the spectral response of the intervening fluid.

In accordance with one aspect of the present invention, the spectral sources are individual wavelengths. The spectral sources can also be collections of wavelengths. They can also be combinations of individual wavelengths and multiple wavelength or continuous spectral sources corresponding to spectral regions of interest. The spectral sources are chosen to obtain spectral information pertaining to the intervening fluid or the vessel walls.

In accordance with a further aspect of the present invention, the spectral sources are modulated in amplitude at a unique frequency. They can also be modulated in amplitude with a unique signature allowing for separation of respective spectral signals. The unique signature can be a collection of modulation frequencies and can further be a digital orthogonal code. The unique signature on each spectral source is preferably largely uncorrelated with all of the other spectral source signatures. Further, the unique signature on each spectral source is preferably the result of a random or pseudo-random process.

The step of separating the spectral signals is preferably performed using a correlation process involving the correlation of the reflected signal amplitude with known reference signals corresponding to the amplitude modulation on each spectral component of the illumination. In accordance with another aspect of the present invention, the correlation process is performed using coherent detection of the amplitude signals. Further, the correlation process can be performed using discrete electronic coherent detection components or digital signal processing techniques.

The step of separating the spectral signals can also be performed using filtering and a correlation process involving the correlation of the reflected signal amplitude with known reference signals corresponding to the amplitude modulation on each spectral component of the illumination.

In accordance with another aspect of the present invention, the reflected light is collected and transported out of the catheter back to a separate detection system as light. The reflected light can be collected in a detector situated in the probe and transported out of the catheter back to a separate detection system as an electrical signal.

In accordance with a preferred embodiment of the present invention, the differences in the impairment due to the intervening fluid varies across the spectrum and is removed through a processing of the correlated of spectral information between the individual spectral regions.

In the present invention, a subject can be diagnosed in vitro as having blood vessel obstructions, atherosclerosis or arterial lesions. The subject being diagnosed can be a mammal.

The present invention also provides a system for optically examining blood vessel walls with a probe through intervening fluid. The system includes a probe to illuminate the vessel walls with multiple spectral sources, a detector to receive optical signals from the probe and a processor to analyze and measure the spectral information.

In accordance with one aspect of the system, the intervening fluid is blood.

The optical illumination from the probe can be comprised of multiple spectral sources each with a unique amplitude modulation allowing for the separation of spectral information subsequent to reflection from the vessel walls. The multiple spectral sources can be each at individual wavelengths. The multiple spectral sources can also each be collections of wavelengths.

In accordance with a further aspect of the present invention, the optical signals are carried by optical fibers.

In accordance with a further aspect of the present invention, the probe has a detector placed inside it.

In accordance with a further aspect of the present invention, the multiple spectral sources are combined to form a single optical signal. Alternatively, the single optical signal is used to probe vessel walls.

In accordance with a further aspect of the present invention, the detector receives a portion of the composite signal and converts it into an electrical signal.

In accordance with a further aspect of the present invention, the electrical signals are carried by electrical wires.

The electrical signal emerging from the detector can be comprised of a collection of carriers at different frequencies. The electrical signal can be carried to one or more amplifiers prior to filtering. Alternatively, the electrical signal can be carried into a series of filters, each filter having its own reference signal frequency.

In accordance with another aspect of the present invention, the electrical signal is passed into a mixer which performs a multiplication of the electrical signal and the reference signal from the modulating frequency sources. The output of the mixer contains a DC level proportional to the amplitude of the electrical carrier at the frequency corresponding to the reference signal frequency. The mixer can also perform a multiplication of the electrical signal and a reference signal with a frequency different from the modulating frequency sources by a prescribed offset corresponding to an intermediate frequency (IF). The output of the mixer can also contain an electrical signal at a prescribed intermediate frequency (IF) whose amplitude is proportional to the amplitude of the electrical carrier at the frequency corresponding to the reference signal frequency. The output of the mixer can be passed on to a processor for further analysis of the spectral information.

In accordance with another aspect of the present invention, a single optical signal can be connected to an optical splitter. The optical splitter splits the composite optical source into separate measurement and detection systems.

The present invention also provides a system for measuring plaque in arteries, the system including a plurality of optical sources, a plurality of frequency sources, each one of the plurality of frequency sources having a different frequency and being connected to one of the plurality of optical sources to modulate the output of each one of the plurality of optical sources, a wavelength division multiplexer that receives the output of each one of the plurality of optical sources and forms an optical output signal, an optical fiber that can conduct the optical output signal and that can receive a reflected optical signal, an optical detector that can detect the reflected optical signal, a plurality of bandpass filters, each one of the plurality of bandpass filters having a passband related to one of the plurality of frequency sources, and a plurality of demodulators, each one of the plurality of demodulators being connected to one of the plurality of bandpass filers.

The processor can be connected to a plurality of demodulators.

A display can be connected to the processor. A storage device can be connected to the processor.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

What is claimed is:

1. A method for optically examining blood vessel walls with a probe through intervening fluid, the method comprising:
simultaneously illuminating the vessel walls with multiple spectral sources;
receiving optical signals from the vessel walls through the intervening fluid at the probe; and
analyzing the optical signals to determine the wavelength dependency of the reflectivity of the vessel wall.

2. A method as claimed in claim 1, wherein the optical illumination from the probe is comprised of multiple spectral sources each with a unique amplitude modulation allowing for the separation of spectral information subsequent to reflection from the vessel walls.

3. A method as claimed in claim 1, wherein the step of analyzing the optical signals comprises the separation of the different spectral signals and determining their respective amplitudes.

4. A method as claimed in claim 1, wherein the step of analyzing the optical signals comprises analyzing a spectral response of the optical signals based on spectral features of the intervening fluid.

5. A method as claimed in claim 2, wherein the spectral sources are modulated in amplitude at a unique frequency.

6. A method as claimed in claim 2, wherein the spectral sources are modulated in amplitude with a unique signature allowing for separation of respective spectral signals.

7. A method as claimed in claim 6, wherein the unique signature is a collection of modulation frequencies.

8. A method as claimed in claim 6, wherein the unique signature is a digital orthogonal code.

9. A system for optically examining blood vessel walls with a probe through intervening fluid, the system comprising:
a probe to illuminate the vessel walls with multiple spectral sources;
a detector to receive optical signals from the probe; and
a processor to analyze and measure the spectral information.

10. The system of claim 9, wherein the optical illumination from the probe is comprised of multiple spectral sources each with a unique amplitude modulation allowing for the separation of spectral information subsequent to reflection from the vessel walls.

11. The system of claim 10, wherein the multiple spectral sources are each at individual wavelengths.

12. The system of claim 10, wherein the multiple spectral sources are each collections of wavelengths.

13. The system of claim 9, wherein the probe has the detector inside it.

14. The system of claim 9, wherein the multiple spectral sources are combined to form a single optical signal.

15. The system of claim 9, wherein the electrical signal emerging from the detector is comprised of a collection of carriers at different frequencies, further comprising a series of filters connected to the detector, each filter having its own reference signal frequency a mixer connected to the series of filters and a processor connected to the mixer.

16. The system of claim 15, wherein the processor analyzes spectral information.

17. A system for measuring plaque in arteries, comprising:
a plurality of optical sources;
a plurality of frequency sources, each one of the plurality of frequency sources having a different frequency and being connected to one of the plurality of optical sources to modulate the output of each one of the plurality of optical sources;
a wavelength division multiplexer that receives the output of each one of the plurality of optical sources and forms an optical output signal;
an optical fiber that can conduct the optical output signal and that can receive a reflected optical signal;
an optical detector that can detect the reflected optical signal;
a plurality of bandpass filters, each one of the plurality of bandpass filters having a passband related to one of the plurality of frequency sources; and
a plurality of demodulators, each one of the plurality of demodulators being connected to one of the plurality of bandpass filers.

18. The system as claimed in claim 17, further comprising a processor connected to the plurality of demodulators.

19. The system as claimed in claim 18, further comprising a display connected to the processor.

20. The system as claimed in claim 18, further comprising a storage device connected to the processor.
